(19) United States
(12) Patent Application Publication
(10) Pub. No.: US 2022/0137431 A1
(43) Pub. Date: May 5, 2022

(54) METHODS AND SYSTEMS FOR PROVIDING LENS FABRICATION DESIGN CAPABLE OF COMPENSATING FOR HIGHER ORDER ABERRATIONS

(71) Applicant: Ovitz Corporation, Rochester, NY (US)

(72) Inventor: Nicolas Scott BROWN, Rochester, NY (US)

(21) Appl. No.: 17/515,226

(22) Filed: Oct. 29, 2021

Related U.S. Application Data
(60) Provisional application No. 63/108,125, filed on Oct. 30, 2020.

(51) Int. Cl. G02C 7/02

(52) U.S. Cl. CPC G02C 7/027; A61B 3/0025; G02C 7/028

(57) ABSTRACT

A method includes receiving information representing a profile of a contact lens capable of compensating for lower order aberrations of an eye of a patient; accessing information representing higher order aberrations of the eye; generating information representing a profile of a contact lens capable of compensating for the lower order aberrations and the higher order aberrations of the eye based on the information representing the profile of the contact lens capable of compensating for the lower order aberrations of the eye and the information representing the higher order aberrations of the eye; and providing the information representing the profile of the contact lens capable of compensating for the lower order aberrations and the higher order aberrations of the eye, for fabrication of the contact lens capable of compensating for the lower order aberrations and the higher order aberrations of the eye.

### RELATED APPLICATION

This application claims the benefit of, and priority to, U.S. Provisional Patent Application Ser. No. 63/108,125, filed Oct. 30, 2020, which is incorporated by reference herein in its entirety.

### TECHNICAL FIELD

This relates generally to methods for making a contact lens, and particularly to methods for providing lens fabrication design capable of correction of high-order aberrations.

### BACKGROUND

Eyes are important organs, which play a critical role in human's visual perception. An eye has a roughly spherical shape and includes multiple elements, such as cornea, lens, vitreous humour, and retina. Imperfections in these components can cause reduction or loss of vision. For example, too much or too little optical power in the eye (e.g., near-sightedness or far-sightedness) and astigmatism can lead to blurring of the vision.

Corrective lenses (e.g., glasses and contact lenses) are frequently used to compensate for blurring caused by too much or too little optical power and/or astigmatism. However, when eyes have higher-order aberrations (e.g., aberrations higher than astigmatism in the Zernike polynomial model of aberrations, such as coma, spherical aberration, trefoil, quadrafoil, etc.), conventional corrective lenses have not been effective at compensating for all of the aberrations associated with the eyes, resulting in blurry images even when corrective lenses are used.

### SUMMARY

Accordingly, there is a need for corrective lenses that can compensate for higher-order aberrations. However, there is a variation in the structure and orientation of an eye among patients (and even between different eyes of a same patient), and thus, a contact lens placed on an eye will settle in different positions and orientations for different patients (or different eyes). Proper alignment of the corrective lens to the patient's eye is required in order to provide an accurate correction or compensation of the higher-order aberrations in the eye. In addition, high order aberrations vary among different eyes. For example, even a left eye and a right eye of a same person may have different high order aberrations. Thus, a contact lens made for a particular eye (e.g., based on the position information, such as lateral displacements and orientation, as well as vision information, such as high order aberrations, for the eye) is required for effective correction or compensation of the higher-order aberrations in the eye. Furthermore, utilizing the position information and the vision information requires adapting proprietary lens design software to address high-order aberrations based on wavefront or other aberrometer measurements. This arrangement requires each lens software provider to engage individually with those skilled in acquiring higher-order aberration measurements to determine how those measurements are recorded and accessed in order to integrate these measurements with the lower-order aberration prescription and use them to modify lens designs. Integration of the higher-order aberration measurement data to lens fabrication programming in any particular case can require significant re-working of programmed instructions and data in order to generate the desired lens design for an individual patient with detected higher-order aberrations.

The above deficiencies and other problems associated with conventional methods are reduced or eliminated by methods and devices described herein. Such improvements to the lens design methods can reduce cost, simplify workflow, and standardize steps for higher-order aberration correction, which, in turn, would be highly beneficial for patients, clinicians, and lens manufacturers.

In accordance with some embodiments, a method performed by an electronic device with one or more processors and memory includes receiving information representing a profile of a contact lens capable of compensating for lower order aberrations of an eye of a patient; accessing information representing higher order aberrations of the eye of the patient, the information representing higher order aberrations of the eye of the patient including information identifying an alignment of the higher order aberrations of the eye of the patient relative to the profile of the contact lens capable of compensating for the lower order aberrations of the eye of the patient; generating information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and providing the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

In accordance with some embodiments, an electronic device includes one or more processors; and memory storing instructions, which, when executed by the one or more processors, cause the one or more processors to: receive information representing a profile of a contact lens capable of compensating for lower order aberrations of an eye of a patient; access information representing higher order aberrations of the eye of the patient, the information representing higher order aberrations of the eye of the patient including information identifying alignment of the higher order aberrations of the eye of the patient relative to the profile of the contact lens capable of compensating for the lower order aberrations of the eye of the patient; generate information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and provide the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

In accordance with some embodiments, a computer readable storage medium stores one or more programs for execution by one or more processors, the one or more programs including instructions for: receiving information representing a profile of a contact lens capable of compensating for lower order aberrations of an eye of a patient; accessing information representing higher order aberrations of the eye of the patient, the information representing higher order aberrations of the eye of the patient including information identifying alignment of the higher order aberrations of the eye of the patient relative to the profile of the contact lens capable of compensating for the lower order aberrations of the eye of the patient; generating information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and providing the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

In accordance with some embodiments, a method performed by an electronic device with one or more processors and memory includes receiving the information, representing the profile of the contact lens capable of compensating for the lower order aberrations and the higher order aberrations of the eye of the patient, generated by any method described herein; and operating a fabrication apparatus in communication with the electronic device based on the information representing the profile of the contact lens capable of compensating for the lower order aberrations and the higher order aberrations of the eye of the patient to fabricate the contact lens capable of compensating for the lower order aberrations and the higher order aberrations of the eye of the patient.

In accordance with some embodiments, an electronic device includes one or more processors; and memory storing one or more instructions, which, when executed by the one or more processors, cause the one or more processors to perform any method described herein.

In accordance with some embodiments, a computer readable storage medium stores one or more programs for execution by one or more processors, the one or more programs including instructions for performing any method described herein.

Thus, the disclosed methods and devices facilitate modification of a lens profile position information for contact lenses, which can be used to accurately determine a position of a position reference point (e.g., a visual axis) of an eye relative to a contact lens (or vice versa), in conjunction with vision information. Such information, in turn, allows design and manufacturing of customized (e.g., personalized) contact lenses that can compensate for higher-order aberrations in a particular eye.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A is a schematic diagram showing a system for vision characterization in accordance with some embodiments.

FIGS. 1B and 1C illustrate optical components of an optical device in accordance with some embodiments.

FIG. 1D illustrates wavefront sensing with the optical device shown in FIGS. 1B and 1C, in accordance with some embodiments.

FIG. 1E illustrates imaging with the optical device shown in FIGS. 1B and 1C, in accordance with some embodiments.

FIGS. 1F and 1G illustrate optical components of an optical device in accordance with some other embodiments.

FIG. 1H is a front view of a measurement instrument in accordance with some embodiments.

FIG. 2 is a block diagram illustrating electronic components of an optical device in accordance with some embodiments.

FIGS. 3A-3D are schematic diagrams illustrating correction of higher-order aberrations in accordance with some embodiments.

FIG. 3E is a schematic diagram illustrating a perspective view of an eye and aspects of lens positioning that relate to design and fitting of the scleral contact lens.

FIG. 3F is a schematic diagram illustrating a plan view of the eye and the lens shown in FIG. 3E, taken along the visual axis.

FIG. 3G shows an image of a reference lens with marks in accordance with some embodiments.

FIG. 4 is a flow diagram illustrating a method of forming a contact lens in accordance with some embodiments.

FIG. 5A shows a process for generating a lens fabrication file in accordance with some embodiments.

FIG. 5B shows a modular process for generating a lens fabrication file in accordance with some embodiments.

FIG. 6 is a schematic diagram illustrating aspects of the lens design file for expressing height data.

FIG. 7 is an excerpt from a lens design file in accordance with some embodiments.

FIG. 8 shows an arrangement for an HOA data file in accordance with some embodiments.

FIGS. 9A and 9B are flow diagrams illustrating a method of providing profile information for a contact lens capable of compensating for higher-order aberrations in accordance with some embodiments.

FIG. 10 is a flow diagram illustrating a method of fabricating a contact lens capable of compensating for higher-order aberrations in accordance with some embodiments.

These figures are not drawn to scale unless indicated otherwise.

### DETAILED DESCRIPTION

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first image sensor could be termed a second image sensor, and, similarly, a second image sensor could be termed a first image sensor, without departing from the scope of the various described embodiments. The first image sensor and the second image sensor are both image sensors, but they are not the same image sensor.

The terminology used in the description of the embodiments herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

A corrective lens (e.g., contact lens) designed to compensate for higher-order aberrations of an eye needs accurate positioning on an eye. If a corrective lens designed to compensate for higher-order aberrations of an eye is not placed accurately, the corrective lens may not be effective in compensating for higher-order aberrations of the eye and may even exacerbate the higher-order aberrations.

One of the additional challenges is that when a corrective lens (e.g., contact lens) is used to compensate for higher-order aberrations of an eye, an apex of a corrective lens is not necessarily positioned on a visual axis of the eye. Thus, a relative position between the visual axis of the eye and the apex of the corrective lens needs to be reflected in the design of the corrective lens. This requires accurate measurements of the visual axis of the eye and a position of the corrective lens on the eye and fabrication of a corrective lens that compensates for the offset between the position of the visual axis of the eye and the position of the corrective lens. Because the position of the corrective lens on an eye depends largely on the specific structure of the eyeball (e.g., the size and curvature) and the surrounding structure (e.g., eyelids), a corrective lens customized for a particular eye is required so that the correction or compensation pattern of the corrective lens is placed in the correct position. Furthermore, conventional lens design and manufacturing software has not been configured to utilize such information needed for manufacturing customized corrective lenses that compensate for higher-order aberrations of respective eyes.

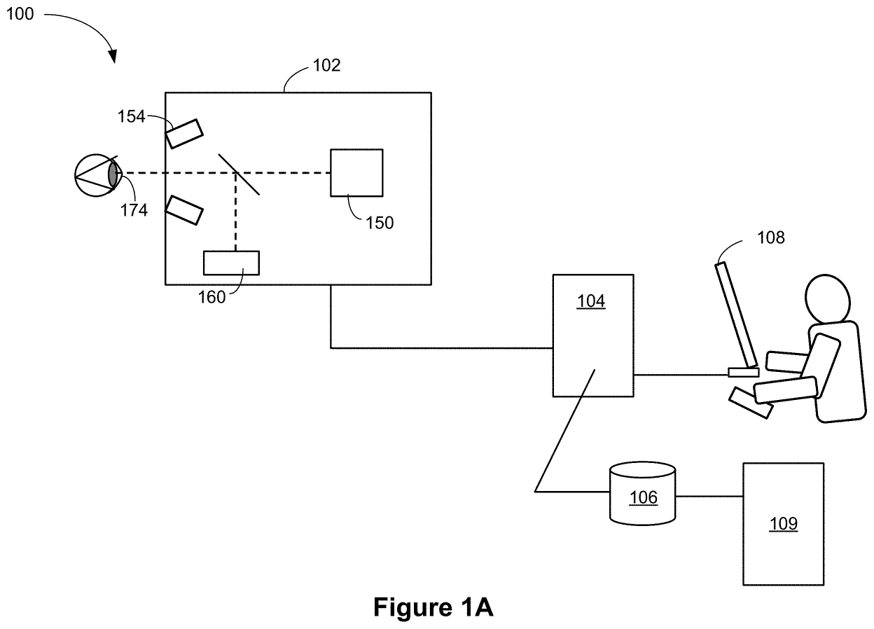

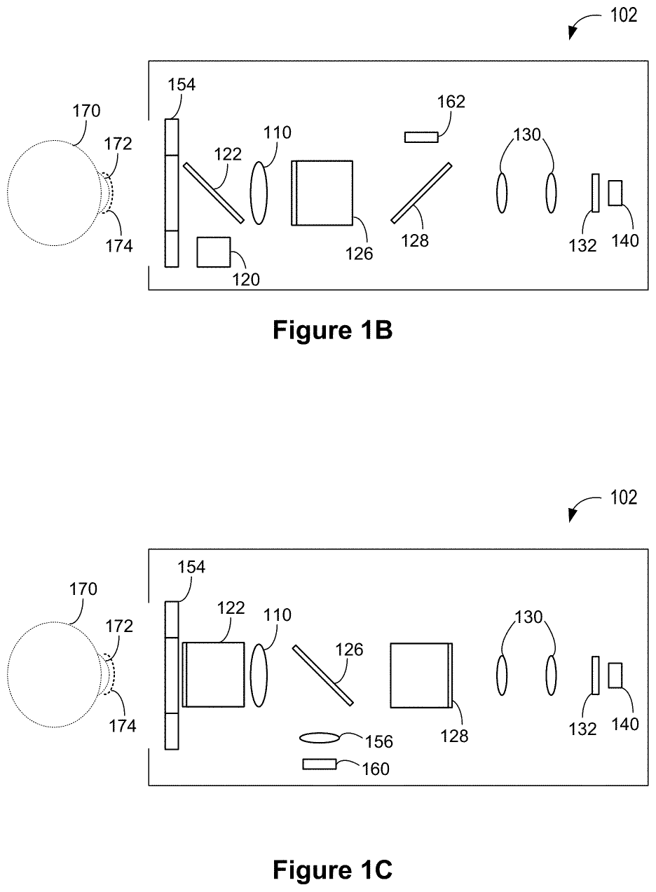

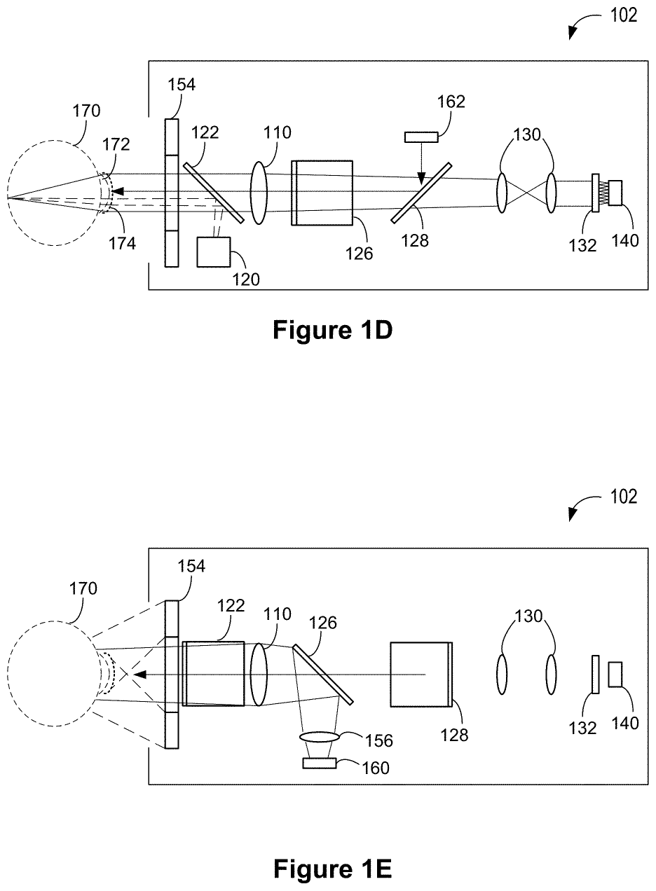

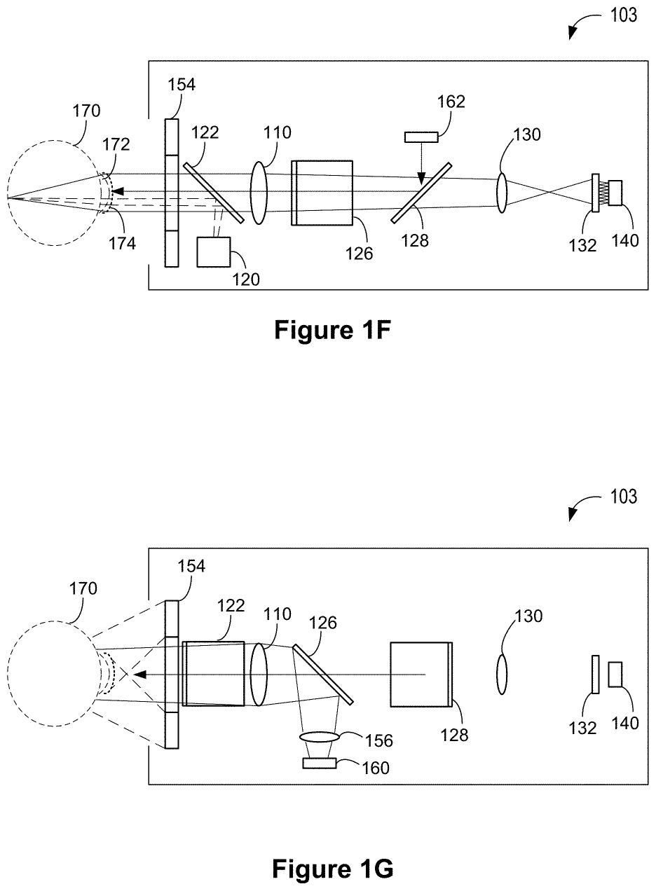

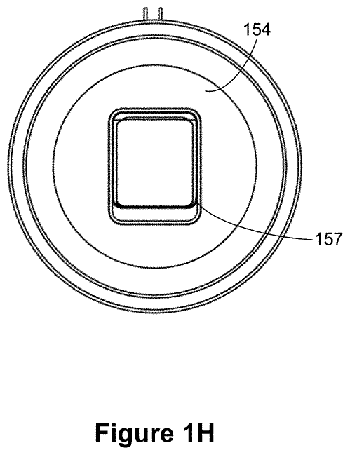

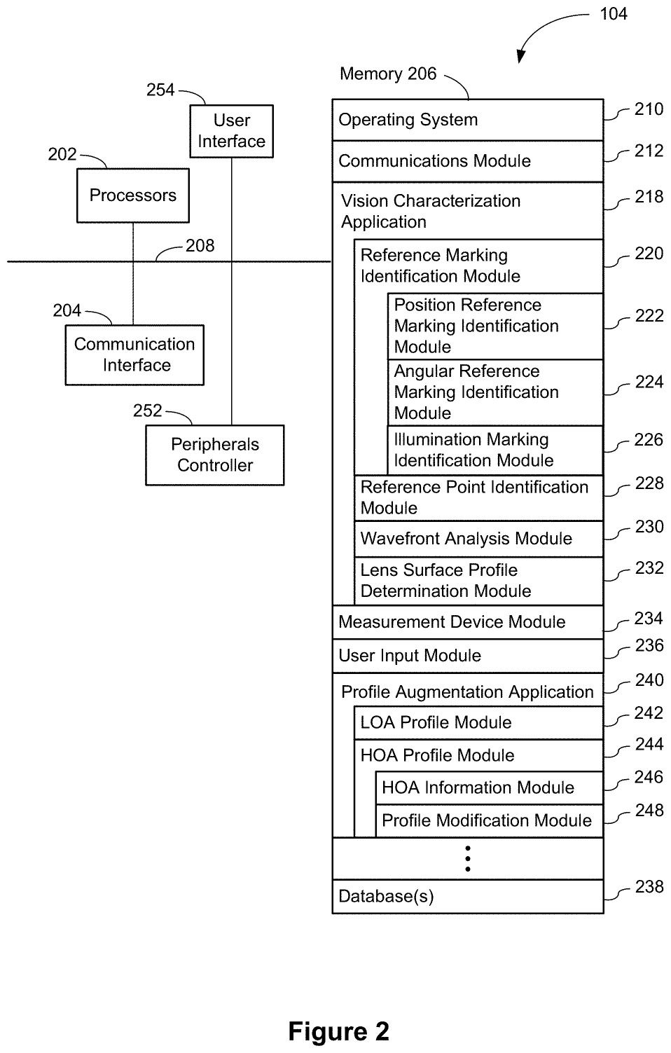

FIG. 1A is a schematic diagram showing a system 100 for vision characterization in accordance with some embodiments. The system 100 includes a measurement device 102, a computer system 104, a database 106, and a display device 108. The measurement device 102 performs a vision characterization of an eye of a patient (e.g., using light source 154 and imaging sensor 160) and provides imaging results and vision profile metrics of the characterized eye. In some cases, the eye is wearing a corrective lens (e.g., a contact lens). The measurement device 102 includes a wavefront measurement device, such as a Shack-Hartmann wavefront sensor 150, that is configured to perform wavefront measurements. The display device 108 shows the imaging results and vision profile metrics acquired by the measurement device 102. The database 106 stores imaging results and vision profile metrics acquired by the measurement device 102. In response to receiving the results from the measurement device 102, the system 100 may generate a correction lens (e.g., contact lens) fabrication file for the patient that is stored in the database 106. In some configurations, the correction lens fabrication file stored in the database 106 is accessed by (or provided to) a fabrication apparatus 109 (e.g., a cutting tool, such as a lathe, a milling machine, a computer numerical control (CNC) system (e.g., a CNC lathe or a CNC turning center), etc.).

The computer system 104 may include one or more computers or central processing units (CPUs). The computer system 104 is in communication with each of the measurement device 102, the database 106, and the display device 108.

FIGS. 1B-1E illustrate optical components of the measurement device 102 in accordance with some embodiments. FIG. 1B shows a side view (e.g., a side elevational view) of the optical components of the measurement device 102, and FIG. 1C is a top view (e.g., a plan view) of the optical components of the measurement device 102. One or more lenses 156 and second image sensor 160 shown in FIG. 1C are not shown in FIG. 1B to avoid obscuring other components of the measurement device 102 shown in FIG. 1B. In FIG. 1C, pattern 162 is not shown to avoid obscuring other components of the measurement device 102 shown in FIG. 1C.

The measurement device 102 includes lens assembly 110. In some embodiments, lens assembly 110 includes one or more lenses. In some embodiments, lens assembly 110 is a doublet lens. For example, a doublet lens is selected to reduce spherical aberration and other aberrations (e.g., coma and/or chromatic aberration). In some embodiments, lens assembly 110 is a triplet lens. In some embodiments, lens assembly 110 is a singlet lens. In some embodiments, lens assembly 110 includes two or more separate lenses. In some embodiments, lens assembly 110 includes an aspheric lens. In some embodiments, a working distance of lens assembly 110 is between 10-100 mm (e.g., between 10-90 mm, 10-80 mm, 10-70 mm, 10-60 mm, 10-50 mm, 15-90 mm, 15-80 mm, 15-70 mm, 15-60 mm, 15-50 mm, 20-90 mm, 20-80 mm, 20-70 mm, 20-60 mm, 20-50 mm, 25-90 mm, 25-80 mm, 25-70 mm, 25-60 mm, or 25-50 mm). In some embodiments, when the lens assembly includes two or more lenses, an effective focal length of a first lens (e.g., the lens positioned closest to the pupil plane) is between 10-150 mm (e.g., between 10-140 mm, 10-130 mm, 10-120 mm, 10-110 mm, 10-100 mm, 10-90 mm, 10-80 mm, 10-70 mm, 10-60 mm, 10-50 mm, 15-150 mm, 15-130 mm, 15-120 mm, 15-110 mm, 15-100 mm, 15-90 mm, 15-80 mm, 15-70 mm, 15-60 mm, 15-50 mm, 20-150 mm, 20-130 mm, 20-120 mm, 20-110 mm, 20-100 mm, 20-90 mm, 20-80 mm, 20-70 mm, 20-60 mm, 20-50 mm, 25-150 mm, 25-130 mm, 25-120 mm, 25-110 mm, 25-100 mm, 25-90 mm, 25-80 mm, 25-70 mm, 25-60 mm, 25-50 mm, 30-150 mm, 30-130 mm, 30-120 mm, 30-110 mm, 30-100 mm, 30-90 mm, 30-80 mm, 30-70 mm, 30-60 mm, 30-50 mm, 35-150 mm, 35-130 mm, 35-120 mm, 35-110 mm, 35-100 mm, 35-90 mm, 35-80 mm, 35-70 mm, 35-60 mm, 35-50 mm, 40-150 mm, 40-130 mm, 40-120 mm, 40-110 mm, 40-100 mm, 40-90 mm, 40-80 mm, 40-70 mm, 40-60 mm, 40-50 mm, 45-150 mm, 45-130 mm, 45-120 mm, 45-110 mm, 45-100 mm, 45-90 mm, 45-80 mm, 45-70 mm, 45-60 mm, 45-50 mm, 50-150 mm, 50-130 mm, 50-120 mm, 50-110 mm, 50-100 mm, 50-90 mm, 50-80 mm, 50-70 mm, or 50-60 mm). In some embodiments, for an 8 mm pupil diameter, the lens diameter is 16-24 mm. In some embodiments, for a 7 mm pupil diameter, the lens diameter is 12-20 mm. In some embodiments, the f-number of lens assembly is between 2 and 5. The use of a common lens assembly (e.g., lens assembly 110) in both a wavefront sensor and a contact lens center sensor allows the integration of the wavefront sensor and the contact lens center sensor without needing large diameter optics.

The measurement device 102 also includes a wavefront sensor. In some embodiments, the wavefront sensor includes first light source 120, lens assembly 110, an array of lenses 132 (also called herein lenslets), and first image sensor 140. In some embodiments, the wavefront sensor includes additional components (e.g., one or more lenses 130). In some embodiments, the wavefront sensor does not include such additional components.

First light source 120 is configured to emit first light and transfer the first light emitted from the first light source toward eye 170, as depicted in FIG. 1D.

FIGS. 1B-1E include eye 170, its components (e.g., cornea 172), and contact lens 174 to illustrate the operations of the measurement device 102 with eye 170 and contact lens 174. By performing measurements on eye 170 with contact lens 174, aberrations in eye 170 as modified by contact lens 174 may be detected. In addition, the position of contact lens 174 relative to eye 170 may be detected. However, eye 170, its components, and contact lens 174 are not part of the measurement device 102.

Turning back to FIG. 1B, in some embodiments, first light source 120 is configured to emit light of a single wavelength or a narrow band of wavelengths. Exemplary first light source 120 includes a laser (e.g., a laser diode) or a light-emitting diode (LED).

In some embodiments, first light source 120 includes one or more lenses to change the divergence of the light emitted from first light source 120 so that the light, after passing through the one or more lenses, is collimated.

In some embodiments, first light source 120 includes a pinhole (e.g., having a diameter of 1 mm or less, such as 400 μm, 500 μm, 600 μm, 700 μm, 800 μm, 900 μm, and 1 mm).

In some cases, an anti-reflection coating is applied on a back surface (and optionally, a front surface) of lens assembly 110 to reduce reflection. In some embodiments, first light source 120 is configured to transfer the first light emitted from first light source 120 off an optical axis of the measurement device 102 (e.g., an optical axis of lens assembly 110), as shown in FIG. 1D (e.g., the first light emitted from first light source 120 propagates parallel to, and offset from, the optical axis of lens assembly 110). This reduces back reflection of the first light emitted from first light source 120, by cornea 172, toward first image sensor 140. In some embodiments, the wavefront sensor includes a quarter-wave plate to reduce back reflection, of the first light, from lens assembly 110 (e.g., light reflected from lens assembly 110 is attenuated by the quarter-wave plate). In some embodiments, the quarter-wave plate is located between beam steerer 122 and first image sensor 140.

First image sensor 140 is configured to receive light, from eye 170, transmitted through lens assembly 110 and the array of lenses 132. In some embodiments, the light from eye 170 includes light scattered at a retina or fovea of eye 170 (in response to the first light from first light source 120). For example, as shown in FIG. 1D, light from eye 170 passes multiple optical elements, such as beam steerer 122, lens assembly 110, beam steerer 126, beam steerer 128, and lenses 130, and reaches first image sensor 140.

Beam steerer 122 is configured to reflect light from light source 120 and transmit light from eye 170, as shown in FIG. 1D. Alternatively, beam steerer 122 is configured to transmit light from light source 120 and reflect light from eye 170. In some embodiments, beam steerer 122 is a beam splitter (e.g., 50:50 beam splitter, polarizing beam splitter, etc.). In some embodiments, beam steerer 122 is a wedge prism, and when first light source 120 is configured to have a linear polarization, the polarization of the light emitted from first light source 120 is configured to reflect at least partly by the wedge prism. Light of a polarization that is orthogonal to the linear polarization of the light emitted from first light source 120 is transmitted through the wedge prism. In some cases, the wedge prism also reduces light reflected from cornea 172 of eye 170.

In some embodiments, beam steerer 122 is tilted at such an angle (e.g., an angle between the optical axis of the measurement device 102 and a surface normal of beam steerer 122 is at an angle less than 45°, such as 30°) so that the space occupied by beam steerer 122 is reduced.

In some embodiments, the measurement device 102 includes one or more lenses 130 to modify a working distance of the measurement device 102.

The array of lenses 132 is arranged to focus incoming light onto multiple spots, which are imaged by first image sensor 140. As in Shack-Hartmann wavefront sensor, an aberration in a wavefront causes displacements (or disappearances) of the spots on first image sensor 140. In some embodiments, a Hartmann array is used instead of the array of lenses 132. A Hartmann array is a plate with an array of apertures (e.g., through-holes) defined therein.

In some embodiments, one or more lenses 130 and the array of lenses 132 are arranged such that the wavefront sensor is configured to measure a reduced range of optical power. A wavefront sensor that is capable of measuring a wide range of optical power may have less accuracy than a wavefront sensor that is capable of measuring a narrow range of optical power. Thus, when a high accuracy in wavefront sensor measurements is desired, the wavefront sensor can be designed to cover a narrow range of optical power. For example, a wavefront sensor for diagnosing low and medium myopia can be configured with a narrow range of optical power between 0 and −6.0 diopters, with its range centering around −3.0 diopters. Although such a wavefront sensor may not provide accurate measurements for diagnosing hyperopia (or determining a prescription for hyperopia), the wavefront sensor would provide more accurate measurements for diagnosing myopia (or determining a prescription for myopia) than a wavefront sensor that can cover both hyperopia and myopia (e.g., from −6.0 to +6.0 diopters). In addition, there are certain populations in which it is preferable to maintain a center of the range at a non-zero value. For example, in some Asian populations, the optical power may range from +6.0 to −14.0 diopters (with the center of the range at −4.0 diopters), whereas in some Caucasian populations, the optical power may range from +8.0 to −12.0 diopters (with the center of the range at −2.0 diopters). The center of the range can be shifted by moving the lenses (e.g., one or more lenses 130 and/or the array of lenses 132). For example, defocusing light from eye 170 can shift the center of the range.

The measurement device 102 further includes a contact lens center sensor (or a corneal vertex sensor). In some embodiments, the contact lens center sensor includes lens assembly 110, second light source 154, and second image sensor 160. In some embodiments, as shown in FIG. 1C, second image sensor 160 is distinct from first image sensor 140. In some embodiments, the wavefront sensor includes additional components that are not included in the contact lens center sensor (e.g., array of lenses 132).

Second light source 154 is configured to emit second light and transfer the second light emitted from second light source 154 toward eye 170. As shown in FIG. 1E, in some embodiments, second light source 154 is configured to transfer the second light emitted from second light source 154 toward eye 170 without transmitting the second light emitted from second light source 154 through lens assembly 110 (e.g., second light from second light source 154 is directly transferred to eye 170 without passing through lens assembly 110).

In some embodiments, the measurement device 102 includes beam steerer 126 configured to transfer light from eye 170, transmitted through lens assembly 110, toward first image sensor 140 and/or second image sensor 160. For example, when the measurement device 102 is configured for wavefront sensing (e.g., when light from first light source 120 is transferred toward eye 170), beam steerer 126 transmits light from eye 170 toward first image sensor 140, and when the measurement device 102 is configured for contact lens center determination (e.g., when light from second light source 154 is transferred toward eye 170), beam steerer 126 transmits light from eye 170 toward second image sensor 160.

Second light source 154 is distinct from first light source 120. In some embodiments, first light source 120 and second light source 154 emit light of different wavelengths (e.g., first light source 120 emits light of 900 nm wavelength, and second light source 154 emits light of 800 nm wavelength; alternatively, first light source 120 emits light of 850 nm wavelength, and second light source 154 emits light of 950 nm wavelength).

In some embodiments, beam steerer 126 is a dichroic mirror (e.g., a mirror that is configured to transmit the first light from first light source 120 and reflect the second light from second light source 154, or alternatively, reflect the first light from first light source 120 and transmit the second light from second light source 154). In some embodiments, beam steerer 126 is a movable mirror (e.g., a mirror that can flip or rotate to steer light toward first image sensor 140 and second image sensor 160). In some embodiments, beam steerer 126 is a beam splitter. In some embodiments, beam steerer 126 is configured to transmit light of a first polarization and reflect light of a second polarization that is distinct from (e.g., orthogonal to) the first polarization. In some embodiments, beam steerer 126 is configured to reflect light of the first polarization and transmit light of the second polarization.

In some embodiments, second light source 154 is configured to project a predefined pattern of light on the eye. In some embodiments, second light source 154 is configured to project an array of spots on the eye. In some embodiments, the array of spots is arranged in a grid pattern.

In some embodiments, second light source 154 includes one or more light emitters (e.g., light-emitting diodes) and diffuser (e.g., a diffuser plate having an array of spots).

FIGS. 1F and 1G illustrate optical components of a measurement instrument 103 in accordance with some other embodiments. Measurement instrument 103 is similar to the measurement device 102 shown in FIGS. 1B-1E except that measurement instrument 103 includes only one lens 130.

FIG. 1H is a front view of the measurement device 102 in accordance with some embodiments. The side view of the measurement device 102 shown in FIG. 1H corresponds to a view of the measurement device 102 seen from a side that is adjacent to second light source 154. In FIG. 1H, the measurement device 102 includes second light source 154, which has a circular shape with a rectangular hole 157 defined in it. Second light source 154 shown in FIG. 1H projects a pattern of light.

Turning back to FIG. 1E, second image sensor 160 is configured to receive light, from eye 170. In some embodiments, the light from eye 170 includes light reflected from cornea 172 of eye 170 (in response to the second light from second light source 154). For example, as shown in FIG. 1E, light from eye 170 (e.g., light reflected from cornea 172) interacts with multiple optical elements, such as lens assembly 110, beam steerer 122, beam steerer 126, and one or more lenses 156, and reaches second image sensor 160.

In some embodiments, the lenses in the contact lens center sensor (e.g., lens assembly 110 and one or more lenses 156) are configured to image a pattern of light projected on cornea 172 onto second image sensor 160.

In some embodiments, second image sensor 160 collects an image of a combination of eye 170 and contact lens 174. From the image, the position and orientation of contact lens 174 relative to eye 170 (e.g., relative to a pupil center or a visual axis of eye 170) may be determined, as described herein.

In some embodiments, the measurement device 102 includes pattern 162 and beam steerer 128. Pattern 162 is an image that is projected toward eye 170 to facilitate positioning of eye 170. In some embodiments, pattern 162 includes an image of an object (e.g., balloon), an abstract shape (e.g., a cross), or a pattern of light (e.g., a shape having a blurry edge).

In some embodiments, beam steerer 128 is a dichroic mirror (e.g., a mirror that is configured to transmit the light from eye 170 and reflect light from pattern 162, or alternatively, reflect light from eye 170 and transmit light from pattern 162). In some embodiments, beam steerer 128 is a movable mirror. In some embodiments, beam steerer 128 is a beam splitter. In some embodiments, beam steerer 128 is configured to transmit light of a first polarization and reflect light of a second polarization that is distinct from (e.g., orthogonal to) the first polarization. In some embodiments, beam steerer 128 is configured to reflect light of the first polarization and transmit light of the second polarization.

FIG. 1D illustrates operation of the measurement device 102 for wavefront sensing without operations for determining a contact lens center and FIG. 1E illustrates operation of the measurement device 102 for determining a contact lens center without wavefront sensing. In some embodiments, the measurement device 102 sequentially operates between wavefront sensing and determining a contact lens center. For example, in some cases, the measurement device 102 performs wavefront sensing and subsequently, determines a contact lens center. In some other cases, the measurement device 102 determines a contact lens center, and subsequently performs wavefront sensing. In some embodiments, the measurement device 102 switches between wavefront sensing and determining a contact lens center. In some embodiments, the measurement device 102 repeats wavefront sensing and determining a contact lens center. In some embodiments, the measurement device 102 operates for wavefront sensing concurrently with determining a contact lens center (e.g., light from first light source 120 and light from second light source 154 are delivered toward eye 170 at the same time, and first image sensor 140 and second image sensor 160 collect images at the same time). For brevity, such details are not repeated herein.

In some embodiments, light from pattern 162 is projected toward eye 170 while the measurement device 102 operates for wavefront sensing (as shown in FIG. 1D). In some embodiments, light from pattern 162 is projected toward eye 170 while device operates for determining a contact lens center (as shown in FIG. 1E).

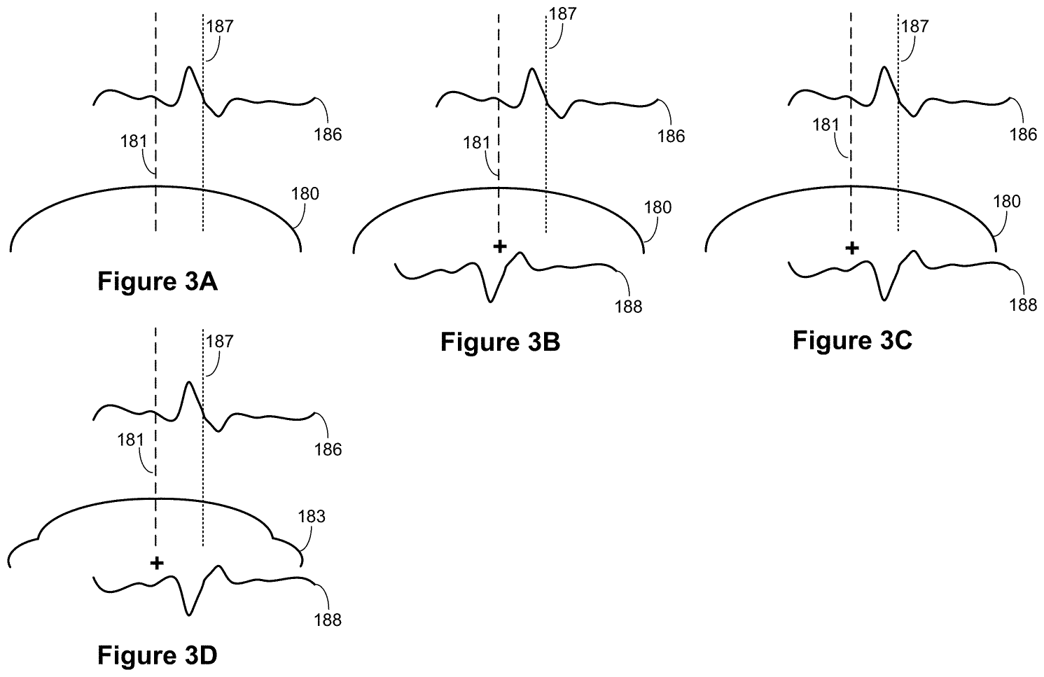

FIG. 2 shows block diagram illustrating electronic components of computer system 104 in accordance with some embodiments. Computer system 104 includes one or more processing units 202 (central processing units, application processing units, application-specific integrated circuit, etc., which are also called herein processors), one or more network or other communications interfaces 204, memory 206, and one or more communication buses 208 for interconnecting these components. In some embodiments, communication buses 208 include circuitry (sometimes called a chipset) that interconnects and controls communications between system components. In some embodiments, system 100 includes a user interface 254 (e.g., a user interface having the display device 108, which can be used for displaying acquired images, one or more buttons, and/or other input devices). In some embodiments, computer system 104 also includes peripherals controller 252, which is configured to control operations of components of the measurement device 102, such as first light source 120, first image sensor 140, second light source 154, and second image sensor 160 (e.g., initiating respective light sources to emit light, and/or receiving information, such as images, from respective image sensors).

In some embodiments, communications interfaces 204 include wired communications interfaces and/or wireless communications interfaces (e.g., Wi-Fi, Bluetooth, etc.).

Memory 206 of computer system 104 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 206 may optionally include one or more storage devices remotely located from the processors 202. Memory 206, or alternately the non-volatile memory device(s) within memory 206, comprises a computer readable storage medium (which includes a non-transitory computer readable storage medium and/or a transitory computer readable storage medium). In some embodiments, memory 206 includes a removable storage device (e.g., Secure Digital memory card, Universal Serial Bus memory device, etc.). In some embodiments, memory 206 or the computer readable storage medium of memory 206 stores the following programs, modules and data structures, or a subset thereof:
operating system 210 that includes procedures for handling various basic system services and for performing hardware dependent tasks;
network communication module (or instructions) 212 that is used for connecting computer system 104 to other computers (e.g., clients and/or servers) via one or more communications interfaces 204 and one or more communications networks, such as the Internet, other wide area networks, local area networks, metropolitan area networks, and so on;
vision characterization application 218 (which may be a stand-alone application or an application that runs in a web browser) that characterizes position information from an image of an eye and markings;
measurement device module 234 that controls operations of the light sources and the image sensors in the measurement device 102 (e.g., for receiving images from the measurement device 102);
user input module 236 configured for handling user inputs on computer system 104 (e.g., pressing of buttons on computer system 104 or pressing of buttons on a user interface, such as a keyboard, mouse, or touch-sensitive display, that is in communication with computer system 104);
profile augmentation application 240 that modifies a profile of a contact lens capable of correcting lower-order aberrations to provide a profile of a contact lens capable of correcting both lower-order aberrations and higher-order aberrations; and
one or more databases 238 (e.g., database 106) that store information acquired by the measurement device 102.

In some embodiments, memory 206 also includes one or both of:
user information (e.g., information necessary for authenticating a user of computer system 104); and
patient information (e.g., optical measurement results and/or information that can identify patients whose optical measurement results are stored in the one or more databases 238 on computer system 104).

In some embodiments, vision characterization application 218 includes the following programs, modules and data structures, or a subset or superset thereof:
reference marking identification module 220 configured for identifying (e.g., automatically identifying) one or more reference markings in an image captured (e.g., recorded, acquired) by the measurement device 102, which may include one or more of the following:
   periphery reference marking identification module 222 configured for identifying (e.g., automatically identifying) one or more periphery reference markings in an image captured (e.g., recorded, acquired) by the measurement device 102;
   angular reference marking identification module 224 configured for identifying (e.g., automatically identifying) one or more angular reference markings in an image captured (e.g., recorded, acquired) by the measurement device 102; and
   illumination marking identification module 226 configured for identifying (e.g., automatically identifying) one or more illumination markings in an image captured (e.g., recorded, acquired) by the measurement device 102;
reference point identification module 228 configured for identifying (e.g., automatically identifying) a position reference point of a patient's eye based on an image captured (e.g., recorded, acquired) by the measurement device 102;
wavefront analysis module 230 configured for analyzing the wavefront measured for a patient's eye(s) using the measurement device 102; and
lens surface profile determination module 232 configured for determining a lens surface profile for a patient's eye(s) based the wavefront measured for a patient's eye and the positions of reference markings.

In some embodiments, wavefront analysis module 230 includes the following programs and modules, or a subset or superset thereof:
an analysis module configured for analyzing images received from first image sensor 140; and
a first presentation module configured for presenting measurement and analysis results from first analysis module (e.g., graphically displaying images received from first image sensor 140, presenting aberrations shown in images received from first image sensor 140, sending the results to another computer, etc.).

In some embodiments, measurement device module 234 includes the following programs and modules, or a subset or superset thereof:
a light source module configured for initiating first light source 120 (through peripherals controller 252) to emit light;
an image sensing module configured for receiving images from first image sensor 140;
a light source module configured for initiating second light source 154 (through peripherals controller 252) to emit light;
an image sensing module configured for receiving images from second image sensor 160;
an image acquisition module configured for capturing one or more images of a patient's eye(s) using the measurement device 102; and
an image stabilization module configured for reducing blurring during acquisition of images by image sensors.

In some embodiments, the computer system 104 may include other modules such as:
an analysis module configured for analyzing images received from second image sensor 160 (e.g., determining a center of a projected pattern of light);
a presentation module configured for presenting measurement and analysis results from second analysis module (e.g., graphically displaying images received from second image sensor 160, presenting cornea curvatures determined from images received from second image sensor 160, sending the results to another computer, etc.);
a spot array analysis module configured for analyzing spot arrays (e.g., measuring displacements and/or disappearances of spots in the spot arrays); and
a centering module configured for determining a center of a projected pattern of light.

In some embodiments, a first image sensing module initiates execution of the image stabilization module to reduce blurring during acquisition of images by first image sensor 140, and a second image sensing module initiates execution of the image stabilization module to reduce blurring during acquisition of images by second image sensor 160.

In some embodiments, a first analysis module initiates execution of spot array analysis module to analyze spot arrays in images acquired by first image sensor 140, and a second analysis module initiates execution of spot array analysis module to analyze spot arrays in images acquired by second image sensor 160.

In some embodiments, a first analysis module initiates execution of spot array analysis module to analyze spot arrays in images acquired by first image sensor 140, and a second analysis module initiates execution of centering module to analyze images acquired by second image sensor 160.

In some embodiments, the one or more databases 238 may store any of: wavefront image data, including information representing the light received by the first image sensor (e.g., images received by the first image sensor), and pupil image data, including information representing the light received by the second image sensor (e.g., images received by the second image sensor).

In some embodiments, profile augmentation application 240 includes the following programs, modules and data structures, or a subset or superset thereof:
lower-order aberrations (LOA) profile module 242, which receives information representing a profile of a contact lens capable of compensating for lower order aberrations of an eye (e.g., from database 238); and
higher-order aberrations (HOA) profile module 242, which generates information representing a profile of a contact lens capable of compensating for both the lower order aberrations and the higher order aberrations of the eye, where the HOA profile module 242 may include one or more of the following:
   HOA information module 246, which accesses information representing higher order aberrations of the eye of the patient; and
   profile modification module 248, which modifies the profile of a contact lens capable of compensating for lower order aberrations of the eye to provide a profile of a contact lens capable of compensating for both the lower order aberrations and the higher order aberrations of the eye.

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, memory 206 may store a subset of the modules and data structures identified above. Furthermore, memory 206 may store additional modules and data structures not described above.

Notwithstanding the discrete blocks in FIG. 2, these figures are intended to be a functional description of some embodiments, although, in some embodiments, the discrete blocks in FIG. 2 can be a structural description of functional elements in the embodiments. One of ordinary skill in the art will recognize that an actual implementation might have the functional elements grouped or split among various components. In practice, and as recognized by those of ordinary skill in the art, items shown separately could be combined and some items could be separated. For example, in some embodiments, measurement device module 234 is part of vision characterization application 218. In other embodiments, reference marking identification module 220, wavefront analysis module 230, and lens surface profile determination module 232 are implemented as separate applications. In some embodiments, one or more programs, modules, or instructions may be implemented in measurement device 102 instead of computer system 104. In some embodiments, the profile augmentation application 240 is implemented in a computer system that is separate from a computer system that includes vision characterization application 218.

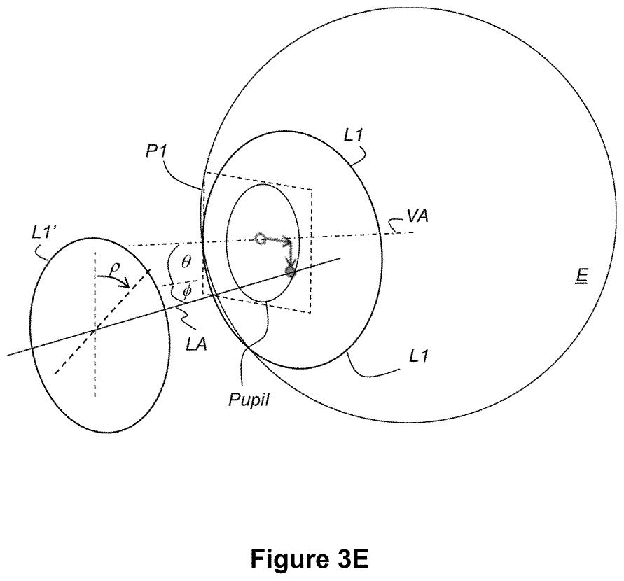

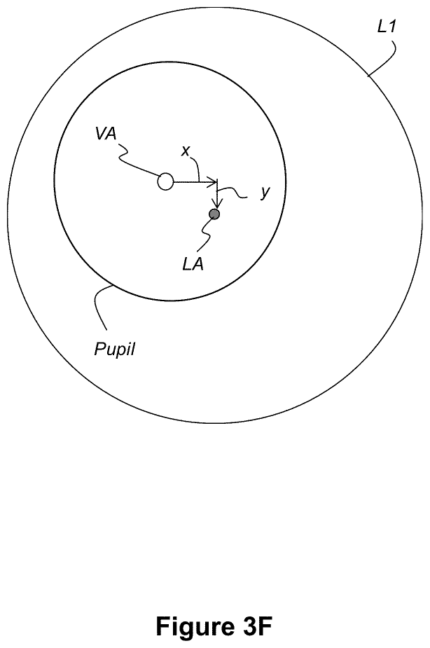

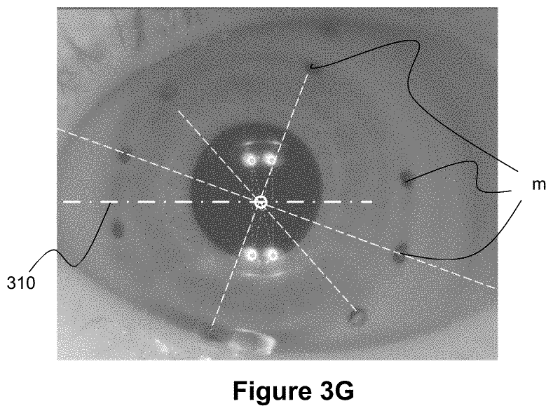

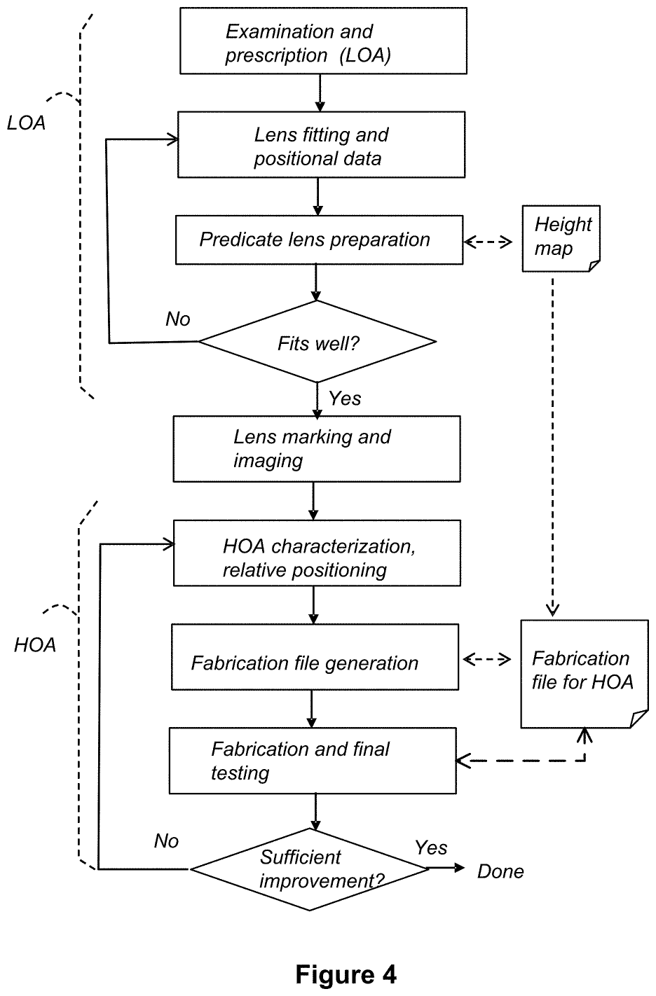

FIGS. 3A-3D are schematic diagrams illustrating correction of higher-order aberrations in accordance with some embodiments.

Unlike focus, higher-order aberrations are highly asymmetric about the optical axis of the patient. Thus, for example, rotation of the higher-order aberration corrected lens out of its intended orientation can render corrective features to be less effective, useless, or even make the patient's vision worse. Positional offset can similarly have negative effects on higher-order aberration correction.

In addition, eye surface shapes can differ from one patient to another. With corrective scleral contact lens, the peripheral portions of the lens seat against the sclera, at a position that can be difficult to predict in all cases. For higher-order aberration correction, it is useful to ascertain, with some accuracy, how the lens rests when placed against the eye early in the process, in order to establish a baseline for close alignment of corrective features.

FIG. 3A illustrates a surface profile of a contact lens 180 without higher-order correction. As a result, an eye wearing the contact lens 180 may see higher-order aberrations represented by line 186. The visual axis 187 of the eye is typically not aligned with the centerline 181 of the contact lens 180, and thus, the measured higher-order aberrations are not aligned with the center of the contact lens 180.

FIG. 3B illustrates modification of the surface profile of the contact lens 180 by superposing a surface profile 188 configured to compensate for the higher-order aberrations. However, when the surface profile 188 is positioned around the centerline 181 of the contact lens 180 as shown in FIG. 3B, the combined surface profile is not effective in reducing the higher-order aberrations, as the surface profile 188 is offset from the higher-order aberrations measured along the visual axis 187 of the eye.

FIG. 3C illustrates modification of the surface profile of the contact lens 180 by superposing the surface profile 188 configured to compensate for the higher-order aberrations where the surface profile 188 is positioned around the visual axis 187 of the eye instead of the centerline 181 of the contact lens 180. By modifying the surface profile of the contact lens 180 by superposing the surface profile 188 with an offset (e.g., the surface profile 188 is in line with the visual axis 187 of the eye), a lens with the modified surface profile can better compensate for higher-order aberrations.

FIG. 3D is similar to FIG. 3C except that the modification of the surface profile can be applied to a multifocal lens 183.

Although FIGS. 3A-3D are used to illustrate the importance of the position of the contact lens relative to the visual axis, the orientation and tilt of the contact lens relative to the visual axis are also important.

FIG. 3E is a schematic diagram illustrating a perspective view of an eye and aspects of lens positioning that relate to design and fitting of the scleral contact lens. FIG. 3F is a schematic diagram illustrating a plan view of the eye and the lens shown in FIG. 3E, taken along the visual axis (e.g., FIG. 3F shows a view of a plane perpendicular to the visual axis).

Coordinates x and y are considered to lie on a plane P1 that is orthogonal to the visual axis VA of the eye E. Angles θ and ϕ, relate to orthogonal angular components for skew of the lens axis LA away from visual axis VA.

Although the lens L1′ is positioned on a surface of the eye E (e.g., over the cornea and sclera), the lens L1′ offset from the surface of the eye E is shown in FIG. 3E to illustrate the rotation of the lens L1 without obscuring other aspects of FIG. 3E. Angle measurement p (also called the orientation) relates to rotation of the lens L1 (e.g., clockwise from a 12 o'clock reference direction). In FIG. 3E, the rotation is measured about the lens axis LA. In some cases, the rotation is measured about the visual axis VA of the eye E.

In some cases, a reference lens with markings is used to assist with determination of the lens position. The reference lens, also called a predicate lens, may serves as an indicator of translation with respect to a visual axis of an eye. In some configurations, the reference lens has a same size as a contact lens (e.g., scleral lens). In some configurations, the reference lens has an optical power (e.g., an optical power to compensate for myopia, hyperopia, or presbyopia, and optionally astigmatism). However, the reference lens may not be configured to compensate for higher-order aberrations. Compared to a contact lens, which is designed to be worn by a patient throughout a day, the reference lens is typically designed to be worn temporarily for diagnostic purposes (e.g., while the patient is at a clinic for one or more measurements by a measurement device, such as measurement device 102, which may be used for prescription of a customized contact lens).

For example, a reference lens with marks, shown in FIG. 3G, may be used to determine a position and orientation of the reference lens while the reference lens is positioned on an eye. As shown in FIG. 3G, the marks m are arranged in a way so that a center of the lens corresponds to a center of the marks m and an orientation of the lens may be indicated by a rotation of the marks m relative to a reference line 310 (e.g., a horizontal line, a vertical line, or a predefined reference line having a particular orientation).

As explained above, the position of a corrective lens on an eye varies among people and even between different eyes of a same person. Thus, a corrective lens customized for a particular eye is required so that the correction or compensation pattern of the corrective lens is placed in the correct position relative to the particular eye.

However, conventional lens designs do not utilize the higher-order aberration information, including the position and orientation of the high-order aberrations relative to (e.g., a center of) the lens. As a result, conventional lens design files, which are used for fabricating contact lenses, do not include such information. Furthermore, conventional lens design software applications are not configured to include the higher-order aberration information, including the position and orientation of the high-order aberrations relative to (e.g., a center of) the lens. Modifying conventional lens design software application to include, or utilize, the higher-order aberration information, including the position and orientation of the high-order aberrations relative to (e.g., a center of) the lens is not a trivial task.

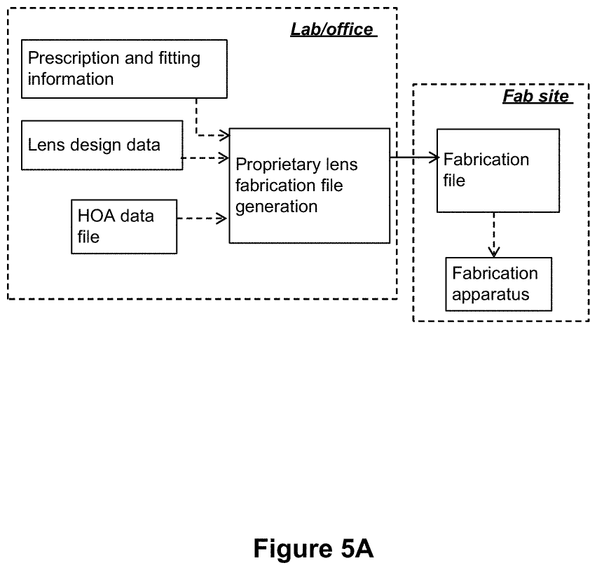

FIG. 4 is a flow diagram illustrating a method of forming a contact lens in accordance with some embodiments.

The process begins with standard examination and prescription performed by a practitioner who identifies a patient as candidate for HOA correction. The basic prescription information for LOA includes standard optical power specification (sphere, cylinder) and adjustment and then can be supplemented by lens fitting information that allows the scleral design to be comfortably and repeatably positionable on the eye surface. Additional information that can be obtained includes scleral surface characterization, lens sizing and diameter factors, coatings or other materials recommendations. This initial lens design data can be generated and stored as lens design data, in a format for lens fabrication as a height map, but without incorporating HOA content. The height map is substantially the lathe data file used for conventional contact lens manufacture.

With the lens design data, the fitting process thus identifies characteristic features of the lens design for forming and testing the fit of a predicate (precursor) lens. Following its fabrication, the predicate lens can have added fiducial markings (e.g., the markings shown in FIG. 3G) that are imaged as part of the fitting process in order to aid in identifying how the lens seats against the eye surface. The fitting must be acceptable before effective HOA characterization can be performed. If the fitting is poor, the lens fitting step must be repeated and results modified accordingly.

Subsequently, HOA characterization using wavefront measurement or other aberrometry data follows the sequence for height map generation and, because higher order aberrations are generally asymmetric, includes information on relative lens positioning. A fabrication file can then be generated using the HOA characterization and positioning data in combination with the LOA lens design data, as described above. Fabrication and final testing steps for the HOA corrective lens follow. In some cases, the fabrication and final testing includes measuring a lens surface profile (e.g., using an interferometer) and comparing the lens surface profile with the fabrication file for HOA (or a surface profile represented by the fabrication file for HOA). After the fabrication and final testing, the patient wears the lens and the vision improvement is tested. If the patient does not indicate vision improvement, it may be useful to repeat steps for HOA characterization or to check for proper equipment setup and generation of the fabrication file. If the patient indicates vision improvement, the process is deemed to be complete.

In some embodiments, a lathe is used as a fabrication apparatus for shaping the outer surface contour of the scleral contact lens in a precision machining process. The lathe is well-adapted for use in shaping surfaces symmetric about an axis, but can also be configured for use in forming asymmetric surface contours. The fabrication file, therefore, gives the fabrication apparatus the necessary spatial coordinates for surface shaping, such as in the form of a height map. The surface contour that is formed on the lens correlates to a refractive distribution or mapping of the measured aberrations of the patient's eye.

In some embodiments, a fabrication apparatus other than a lathe is used to form the scleral lens with the varying refractive properties and refractive distribution that provides HOA correction. For example, a laser-induced lens conditioning device that modifies the refractive properties of localized portions of the lens material after it has been formed, or an additive manufacturing device (e.g., 3D printing device) that can modify refractive properties of the lens as it is manufactured by using a combination of different materials or by adjusting curing treatment over localized regions. The methods described here can also be applied to these other manufacturing devices.

In order to fabricate the final corrective lens for the patient, the measured refractive distribution that has been acquired using HOA measurement must be integrated with the standard prescription that was used for the predicate lens (as shown in the FIG. 4 sequence). Although conventional contact lens design and fabrication software is well suited to the demand for accurate, high production fabrication following standard prescription formulas for LOA correction, integration of the HOA characterization with the standard prescription information is not a straightforward task.

An additional, complicating factor for HOA correction, and not considered for LOA correction in general, relates to lens rotational and translational positioning, as noted earlier. Whereas symmetry (about the optical axis) is a standard feature of conventional contact lenses, lenses adapted for HOA correction have some asymmetry.

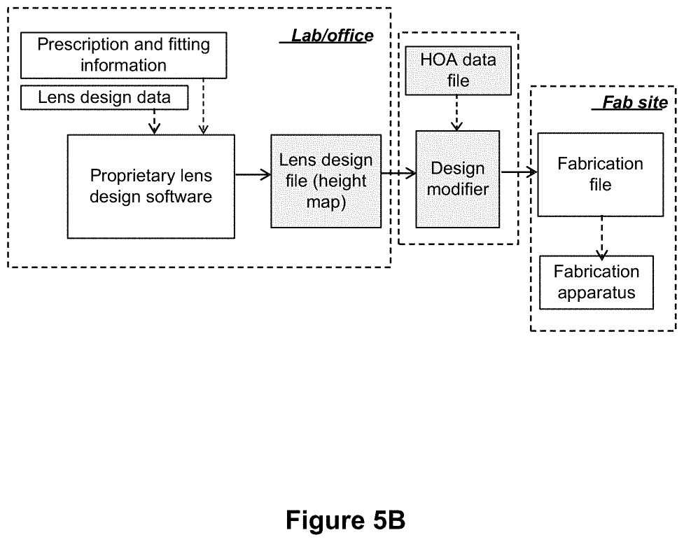

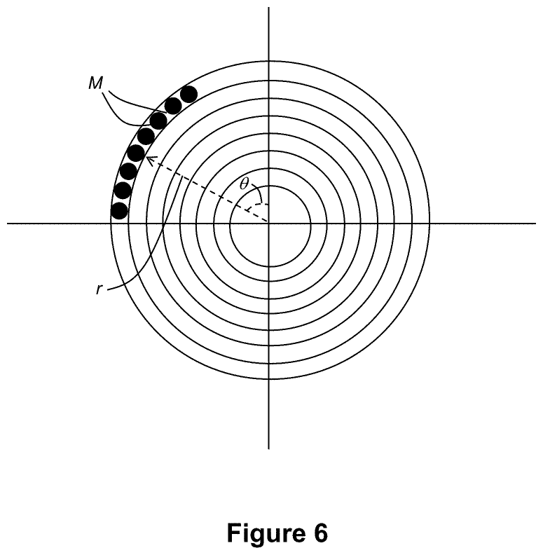

FIG. 5A shows a process for generating a lens fabrication file that includes HOA correction in accordance with some embodiments. In FIG. 5A, prescription and fitting information and an HOA data file are both input to proprietary lens fabrication software, which generates a fabrication file that can be input to the lathe or other fabrication apparatus at a contact lens fabrication site.

In order to make the model of FIG. 5A workable, contact lens design and fabrication software must be modified to incorporate the HOA data file information. As pointed out above, this modification must be able to accommodate both lens positioning and HOA contour or other refractive changes, significantly complicating the task of scleral contact lens fabrication.

Considering this added complexity, the need for software modification in order to integrate HOA fabrication presents a significant burden. Moreover, implementation for different competing systems can mean that, given the same design, final results can differ from one fabrication site to another, depending on the particular software that was used for lens design. This will increase the difficulty in providing consistent and reliable results and lens performance for the HOA patient.

FIG. 5B shows a modular process for generating a lens fabrication file in accordance with some embodiments. In FIG. 5B, the proprietary lens fabrication software does not need to be directly modified in order to integrate lens positioning and HOA refractive correction features. Instead, this software is used in a standard way to generate an initial lens design file, in a standard format for lathe use, but not yet configured for HOA correction.

As described with respect to FIG. 4, the initial lens design file is not a “blank”, but is custom-designed for the individual patient, with care taken to adjust scleral contact and to provide correction for any low-order aberrations (focus, cylinder, astigmatism). For the majority of patients, the lens design file is complete and provides the end-product needed: a contact lens fabricated for LOA correction and not requiring further processing.

For HOA correction, however, the initial lens design file is used to provide a predicate lens, as a type of intermediate. This predicate lens can also be marked for measurement in order to help track lens translation, rotation, tilt and overall seating against the eye; however, the needed measurement for these positioning factors is not performed until the predicate lens is prepared. Wavefront measurements taken through the predicate lens can then more accurately map the refractive distribution of the patient's visual field that relates to HOAs.

There are a limited number of lathe manufacturers and equipment types used for conventional contact lens fabrication. However, there are a larger number of software providers and proprietary software applications designed to generate files used for forming contact lens surfaces or refractive patterns. This design software, from whatever source, is developed to interact with one of a small set of standard fabrication systems. By working with standard output from any commercial or proprietary application, the modular method adapts the output from these software lens design products to generate lens fabrication files that provide HOA correction. The modular method modifies the LOA fabrication file output itself for the lathe or other equipment, and thus requires no change to existing code.

The portion of the workflow sequence labeled for lab/office in FIG. 5B is the conventional workflow for scleral lens design, without consideration of lens positioning and HOAs. The lens design software executes in conventional fashion, generating a predicate lens with the needed LOA correction. Thus, following the FIG. 5B workflow does not impose any new tasks upon the lens design software developer, nor require revision with each improvement of HOA characterization software. The lens design file itself need only provide the information used to construct a scleral contact lens that fits the patient and corrects for the LOAs: vision focus, cylinder, and astigmatism.

At the lab or office site, or some other location, including a networked service location or cloud-based processing resource, a design modifier software application then performs the needed modification of the lens design file, or height map, by combining the design file data from the lab or office with positional and HOA data generated from the measurement apparatus shown in FIG. 1A.

The sequence of FIG. 5B can provide improved consistency over the general method of FIG. 5A, since a single software product can be optimized to provide HOA correction for any lens having its lens design data provided as a fabrication file in suitable format. This allows any third-party software vendor who provides a lens design file that conforms to a given standard to provide what is needed as a starting-point for generating an updated fabrication file that includes HOA correction.

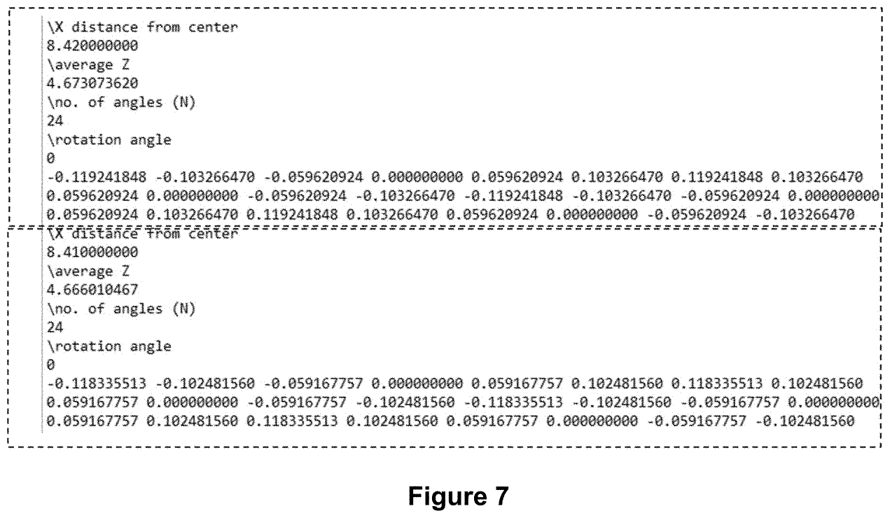

For the conventional contact lens fabrication file, a number of lens design software applications generate a type of height map as an expression of surface contour for lathe fabrication of the contact lens. A physical model behind the underlying data arrangement that has been generally standardized for the height map (or sag map) is shown in coarse detail in FIG. 6. The lens surface mapping can be considered as a series of a number n of concentric circles or rings, each circle having a radius r and a discrete number of angular locations M. Each circle or ring of radius rₙ has an average height value hₙ for its positions M. In the height map file, each location M, at given polar coordinate (θₘ, r_{M}), has a given height h_{M} that is expressed as a positive or negative difference from the average height value hₙ.

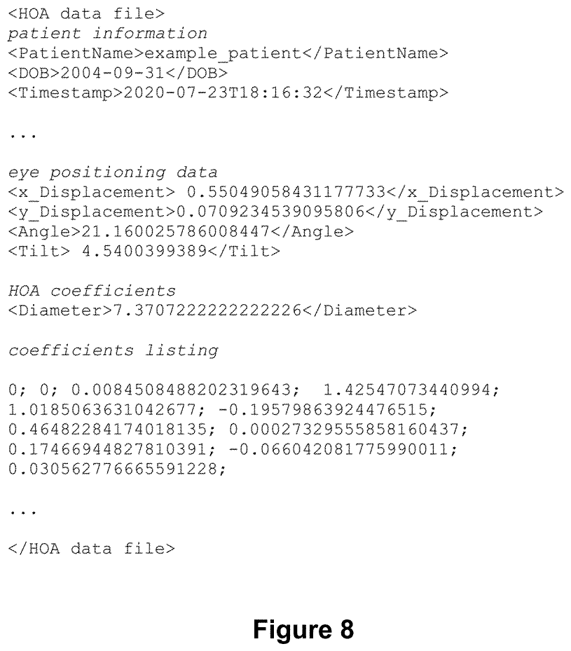

FIG. 7 shows an excerpt from a lens design file in accordance with some embodiments. Data for two among hundreds of circular rings of the lens surface are shown, each ring's data indicated within its own dashed-line box. The radius r is given as X distance from center; values in the example shown are for rings of radii 8.4200000 and 8.410000 mm. Over each radius r, an average height is given as average Z. Then, a number of angular positions (24 in the examples shown) is indicated relative to a reference rotation angle (0°). Each of the 24 positions has a height value, relative to the average Z value. The pattern shown for this small excerpt is repeated for each successive ring of radii r of incrementally smaller size, over the full surface of the lens, providing a mapping that is used to instruct the lathe to dynamically adjust in the Z (axial) direction as it rotates the lens as its workpiece.

As shown in the workflow of FIG. 5B, the lens design file, such as the height map described with reference to FIGS. 6 and 7, can be modified by HOA data obtained by the measurement system shown in FIG. 1A. According to an embodiment, HOA data can be represented in a standard format, such as in an XML file, for example.

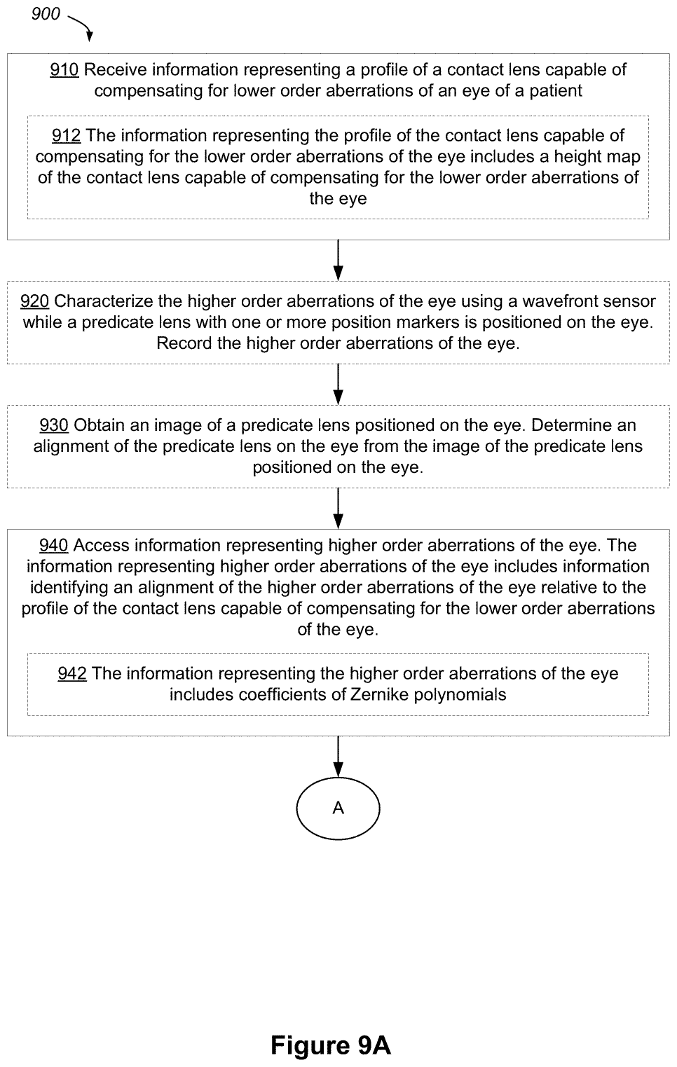

FIG. 8 shows an arrangement for an HOA data file in accordance with some embodiments.

In some embodiments, the HOA data file includes patient identifying information, along with date and time information. In some embodiments, the patient identifying information is encrypted for security and privacy protection.

Positioning data for the eye helps aligning the corrective features of the lens with the corresponding aberration pattern exhibited by the patient.

In some embodiments, the HOA data file includes a pupil diameter and a number of values that show changes within the refractive distribution, such as using Zernike coefficients or other parameters. It can be appreciated that any of a number of data arrangements could be employed for organizing the recorded HOA data.

According to some embodiments, the design modifier software shown in FIG. 5B is configured to accept the lens design file and modify the height data, or other refractive correction data, according to the Zernike coefficients, or similar data measured from the HOA characterization and mapped onto the existing surface data that provides LOA correction. The resulting fabrication file has the format of the lens design file, usable by the lathe equipment, with the added HOA correction.

One benefit of applying the HOA modifications to a separately generated lens design file relates to achieving suitable fitting of the lens against the eye and accurately determining how the lens is positioned, that is, x-y translation, rotation, and tilt. Once these lens data and basic prescription requirements are known and the needed geometry for a functional lens has been established, the lens can be seated on the eye with confidence in its position. HOA mapping can be achieved with enhanced accuracy and the corresponding correction can then be applied with higher probability of success.

A difficulty with HOA-related modifications is that direct application of factors from the wavefront-generated Zernike coefficient data does not necessarily correct vision problems and there can be some cases when attempts to use the generated Zernike coefficients directly can actually cause problems. Thus, for example, slight shifting of the scleral contact lens over the eye surface can inadvertently offset the positioning of an intended correction so that correction areas of the lens are shifted from their intended position and distortion occurs. In some embodiments, the design modifier software application detects instances where correction is impractical for a particular aberration or patient and apply correction only the likelihood of improving patient vision exceeds a certain threshold.

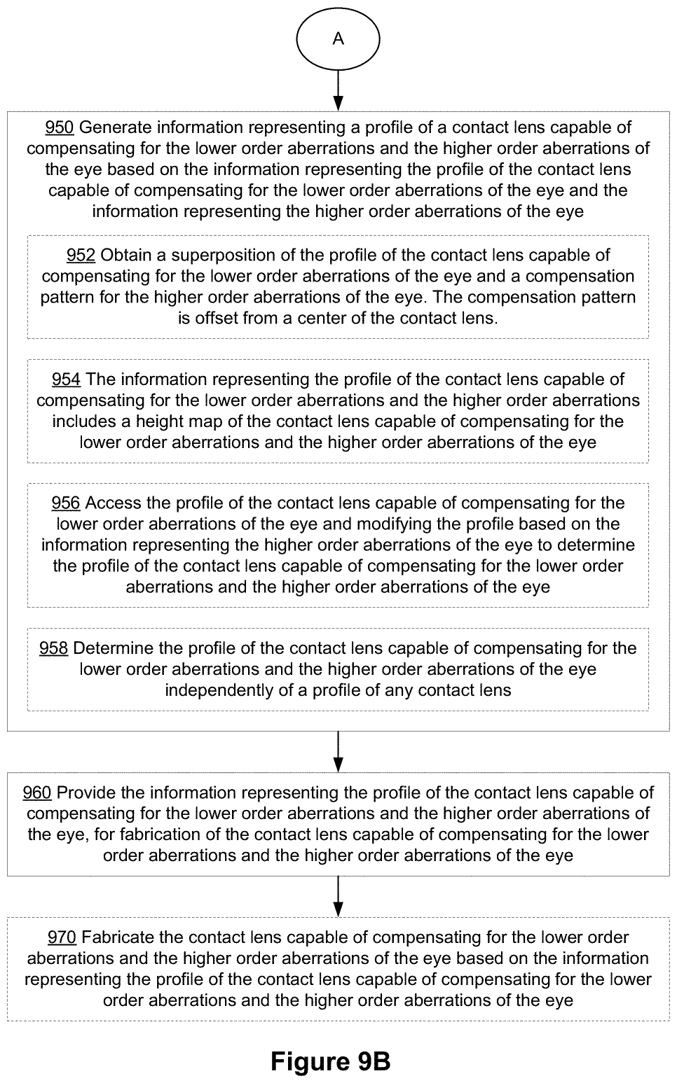

FIGS. 9A and 9B are flow diagrams illustrating a method 900 of providing profile information for a contact lens capable of compensating for higher-order aberrations in accordance with some embodiments. The method 900 is performed by an electronic device with one or more processors and memory (e.g., computer system 104).

The method includes (910) receiving information representing a profile of a contact lens capable of compensating for lower order aberrations of an eye of a patient (e.g., from a lens design software shown in FIG. 5B).

In some embodiments, the information representing the profile of the contact lens capable of compensating for the lower order aberrations of the eye of the patient includes (912) a height map of the contact lens capable of compensating for the lower order aberrations of the eye of the patient (e.g., as shown in FIGS. 6 and 7).

In some embodiments, the method further includes (920) characterizing the higher order aberrations of the eye of the patient using a wavefront sensor while a predicate lens with one or more position markers is positioned on the eye of the patient; and recording the higher order aberrations of the eye of the patient (e.g., as shown in FIG. 1A).

In some embodiments, the method further includes (930) obtaining an image of a predicate lens positioned on the eye of the patient; and determining an alignment of the predicate lens on the eye of the patient from the image of the predicate lens positioned on the eye of the patient (e.g., as shown in FIG. 3G).

The method includes (940) accessing information representing higher order aberrations of the eye of the patient (e.g., as shown in FIG. 5B). The information representing higher order aberrations of the eye of the patient includes information identifying an alignment of the higher order aberrations of the eye of the patient relative to the profile of the contact lens capable of compensating for the lower order aberrations of the eye of the patient.

In some embodiments, the information representing the higher order aberrations of the eye of the patient includes (942) coefficients of Zernike polynomials (e.g., FIG. 8).

The method includes (950) generating information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient (e.g., FIG. 3C).

In some embodiments, generating the information representing the profile of the contact lens capable of compensating for the lower order aberrations and the higher order aberrations of the eye of the patient includes (952) obtaining a superposition of the profile of the contact lens capable of compensating for the lower order aberrations of the eye of the patient and a compensation pattern for the higher order aberrations of the eye of the patient (e.g., FIG. 3C). The compensation pattern is offset from a center of the contact lens.

In some embodiments, the information representing the profile of the contact lens capable of compensating for the lower order aberrations and the higher order aberrations includes (954) a height map of the contact lens capable of compensating for the lower order aberrations and the higher order aberrations of the eye of the patient. For example, the information representing the profile of the contact lens capable of compensating for the lower order aberrations and the higher order aberrations may be stored in the format shown in FIG. 7 based on the scheme illustrated in FIG. 6.

In some embodiments, generating the information representing the profile of the contact lens capable of compensating for the lower order aberrations and the higher order aberrations of the eye of the patient includes (956) accessing the profile of the contact lens capable of compensating for the lower order aberrations of the eye of the patient and modifying the profile based on the information representing the higher order aberrations of the eye of the patient to determine the profile of the contact lens capable of compensating for the lower order aberrations and the higher order aberrations of the eye of the patient (e.g., FIG. 5B).

In some embodiments, generating the information representing the profile of the contact lens capable of compensating for the lower order aberrations and the higher order aberrations of the eye of the patient includes (958) determining the profile of the contact lens capable of compensating for the lower order aberrations and the higher order aberrations of the eye of the patient independently of a profile of any contact lens. Subsequently, the profile of the contact lens capable of compensating for the lower order aberrations and the higher order aberrations of the eye may be superposed on a profile of a contact lens.

The method includes (960) providing the information representing the profile of the contact lens capable of compensating for the lower order aberrations and the higher order aberrations of the eye of the patient, for fabrication of the contact lens capable of compensating for the lower order aberrations and the higher order aberrations of the eye of the patient (e.g., FIG. 4).

In some embodiments, the method further includes (970) fabricating the contact lens capable of compensating for the lower order aberrations and the higher order aberrations of the eye of the patient based on the information representing the profile of the contact lens capable of compensating for the lower order aberrations and the higher order aberrations of the eye of the patient (e.g., FIG. 4).

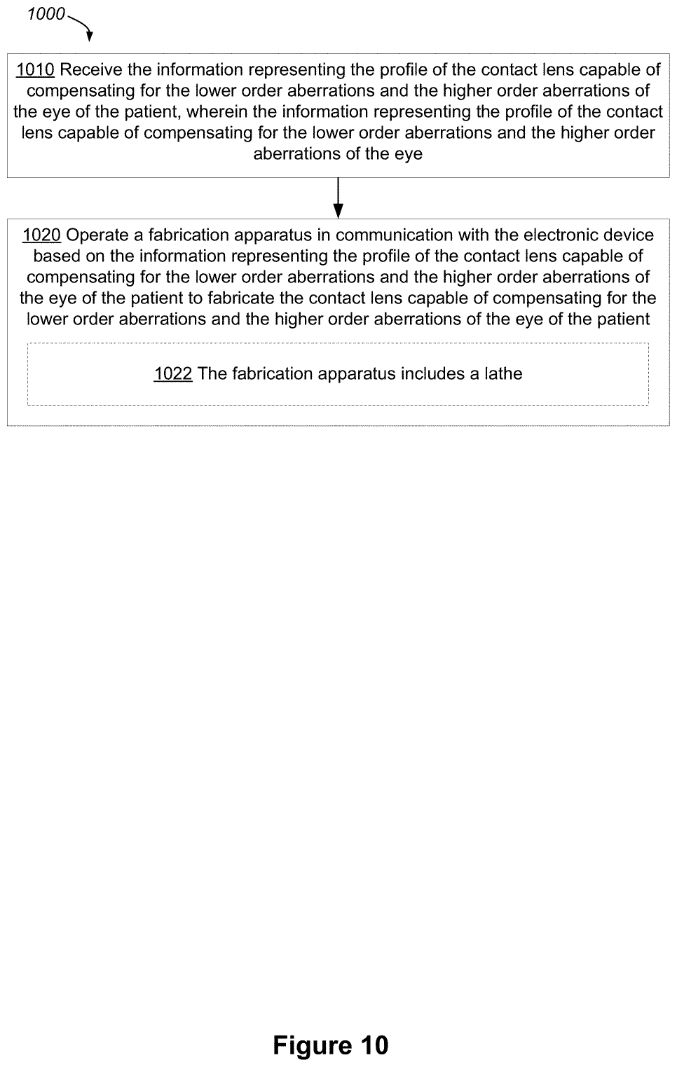

FIG. 10 is a flow diagram illustrating a method 1000 of fabricating a contact lens capable of compensating for higher-order aberrations in accordance with some embodiments. The method 1000 is performed by an electronic device with one or more processors and memory (e.g., a computer system located at a fabrication site).

The method includes (1010) receiving the information representing the profile of the contact lens capable of compensating for the lower order aberrations and the higher order aberrations of the eye of the patient, wherein the information representing the profile of the contact lens capable of compensating for the lower order aberrations and the higher order aberrations of the eye of the patient (e.g., a computer system at a fabrication site receives a modified design file as shown in FIG. 5B).

The method also includes (1020) operating a fabrication apparatus in communication with the electronic device based on the information representing the profile of the contact lens capable of compensating for the lower order aberrations and the higher order aberrations of the eye of the patient to fabricate the contact lens capable of compensating for the lower order aberrations and the higher order aberrations of the eye of the patient.

In some embodiments, the fabrication apparatus includes (1022) a lathe.

In some embodiments, the electronic device is integrated with the fabrication apparatus.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the invention and the various described embodiments with various modifications as are suited to the particular use contemplated.

1. A method performed by an electronic device with one or more processors and memory, the method comprising:
receiving information representing a profile of a contact lens capable of compensating for lower order aberrations of an eye of a patient;
accessing information representing higher order aberrations of the eye of the patient, wherein the information representing higher order aberrations of the eye of the patient includes information identifying an alignment of the higher order aberrations of the eye of the patient relative to the profile of the contact lens capable of compensating for the lower order aberrations of the eye of the patient;
generating information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and
providing the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

2. The method of claim 1, wherein:
the information representing the higher order aberrations of the eye of the patient includes coefficients of Zernike polynomials.

3. The method of claim 1, wherein:
the information representing the profile of the contact lens capable of compensating for the lower order aberrations of the eye of the patient includes a height map of the contact lens capable of compensating for the lower order aberrations of the eye of the patient.

4. The method of claim 1, wherein:
generating the information representing the profile of the contact lens capable of compensating for the lower order aberrations and the higher order aberrations of the eye of the patient includes obtaining a superposition of the profile of the contact lens capable of compensating for the lower order aberrations of the eye of the patient and a compensation pattern for the higher order aberrations of the eye of the patient, wherein the compensation pattern is offset from a center of the contact lens.

5. The method of claim 1, wherein:
the information representing the profile of the contact lens capable of compensating for the lower order aberrations and the higher order aberrations includes a height map of the contact lens capable of compensating for the lower order aberrations and the higher order aberrations of the eye of the patient.

6. The method of claim 1, wherein generating the information representing the profile of the contact lens capable of compensating for the lower order aberrations and the higher order aberrations of the eye of the patient includes accessing the profile of the contact lens capable of compensating for the lower order aberrations of the eye of the patient and modifying the profile based on the information representing the higher order aberrations of the eye of the patient to determine the profile of the contact lens capable of compensating for the lower order aberrations and the higher order aberrations of the eye of the patient.

7. The method of claim 1, wherein generating the information representing the profile of the contact lens capable of compensating for the lower order aberrations and the higher order aberrations of the eye of the patient includes determining the profile of the contact lens capable of compensating for the lower order aberrations and the higher order aberrations of the eye of the patient independently of a profile of any contact lens.

8. The method of claim 1, further comprising:
characterizing the higher order aberrations of the eye of the patient using a wavefront sensor while a predicate lens with one or more position markers is positioned on the eye of the patient; and
recording the higher order aberrations of the eye of the patient.

9. The method of claim 1, further comprising:
obtaining an image of a predicate lens positioned on the eye of the patient; and
determining an alignment of the predicate lens on the eye of the patient from the image of the predicate lens positioned on the eye of the patient.

10. The method of claim 1, further comprising:
fabricating the contact lens capable of compensating for the lower order aberrations and the higher order aberrations of the eye of the patient based on the information representing the profile of the contact lens capable of compensating for the lower order aberrations and the higher order aberrations of the eye of the patient.

11. An electronic device, comprising:
one or more processors; and
memory storing instructions, which, when executed by the one or more processors, cause the one or more processors to:
receive information representing a profile of a contact lens capable of compensating for lower order aberrations of an eye of a patient;
access information representing higher order aberrations of the eye of the patient, wherein the information representing higher order aberrations of the eye of the patient includes information identifying alignment of the higher order aberrations of the eye of the patient relative to the profile of the contact lens capable of compensating for the lower order aberrations of the eye of the patient;
generate information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and
provide the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

12. The electronic device of claim 11, wherein:
the information representing the higher order aberrations of the eye of the patient includes coefficients of Zernike polynomials.

13. A computer readable storage medium storing one or more programs for execution by one or more processors, the one or more programs including instructions for:
receiving information representing a profile of a contact lens capable of compensating for lower order aberrations of an eye of a patient;
accessing information representing higher order aberrations of the eye of the patient, wherein the information representing higher order aberrations of the eye of the patient includes information identifying alignment of the higher order aberrations of the eye of the patient relative to the profile of the contact lens capable of compensating for the lower order aberrations of the eye of the patient;
generating information representing a profile of a contact lens capable of compensating for the lower order aberrations and the higher order aberrations of the eye of the patient based on the information representing the profile of the contact lens capable of compensating for the lower order aberrations of the eye of the patient and the information representing the higher order aberrations of the eye of the patient; and
providing the information representing the profile of the contact lens capable of compensating for the lower order aberrations and the higher order aberrations of the eye of the patient, for fabrication of the contact lens capable of compensating for the lower order aberrations and the higher order aberrations of the eye of the patient.

14. The computer readable storage medium of claim 13, wherein:
the information representing the higher order aberrations of the eye of the patient includes coefficients of Zernike polynomials.

15. A method performed by an electronic device with one or more processors and memory, comprising:
receiving the information representing the profile of the contact lens capable of compensating for the lower order aberrations and the higher order aberrations of the eye of the patient, wherein the information representing the profile of the contact lens capable of compensating for the lower order aberrations and the higher order aberrations of the eye of the patient is generated by the method of claim 1; and
operating a fabrication apparatus in communication with the electronic device based on the information representing the profile of the contact lens capable of compensating for the lower order aberrations and the higher order aberrations of the eye of the patient to fabricate the contact lens capable of compensating for the lower order aberrations and the higher order aberrations of the eye of the patient.

16. The method of claim 15, wherein:
the fabrication apparatus includes a lathe.

17. The method of claim 15, wherein:
the electronic device is integrated with the fabrication apparatus.

18. An electronic device, comprising:
one or more processors; and
memory storing one or more instructions, which, when executed by the one or more processors, cause the one or more processors to perform the method of claim 15.

19. A computer readable storage medium storing one or more programs for execution by one or more processors, the one or more programs including instructions for:
performing the method of claim 15.
